(19) United States
(12) Patent Application Publication
(10) Pub. No.: US 2010/0172986 A1
(43) Pub. Date: Jul. 8, 2010

(54) CONTROLLED RELEASE COMPOSITION AND PREPARATION THEREOF

(75) Inventors: Tong-Ho Lin, Taipei (TW); Kuen-Jen Wang, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: LOTUS PHARMACEUTICAL CO., LTD., TAIPEI (TW)

(21) Appl. No.: 12/304,447

(22) PCT Filed: Jul. 11, 2006
(86) PCT No.: PCT/CN2006/001693

§ 371 Date: Dec. 11, 2008

(51) Int. Cl. A61K 9/28; A61K 47/38; A61K 31/19; A61K 9/00

(52) U.S. Cl. 424/474; 514/772.6; 514/557; 424/400

(57) ABSTRACT

The present invention relates to a controlled release composition comprising hydrophobic cellulose, methacrylic acid copolymer and active ingredient in a form of uniform state. The present invention also relates to a method of preparing a controlled release tablet.

### FIELD OF THE INVENTION

This invention relates to a controlled release composition and a method for producing thereof.

### DESCRIPTION OF PRIOR ART

A sustained release or controlled release drug delivery system can be useful in enhancing patient compliance by reducing the frequency with which medicines need to be administered. A variety of approaches have been used in the art to produce sustained or controlled release drug delivery systems. Such approaches include, for example, either coating a tablet or bead with polymeric material, or making a tablet with insoluble or poorly soluble polymers.

The coating of a tablet or bead, for example, is time consuming, and, as an aqueous coating is usually employed, in general cannot be used when the drug contained in such tablet or bead is moisture sensitive. Likewise, the lot to lot variability of polymers may cause tablets produced solely with insoluble or poorly soluble polymers to exhibit unreproducible performance profiles. (U.S. Pub. No. 20060099254)

U.S. Pat. No. 5,055,306 discloses a sustained-release formulation containing a core having active ingredient, a coating covering substantially the whole surface of the core and comprising water insoluble but water swellable neutral copolymer of ethyl acrylate and methyl methacrylate, and a water soluble hydroxylated cellulose derivative. U.S. Pat. No. 4,952,402 discloses a controlled release powder comprising particles containing an active ingredient in intimate admixture with at least one non-toxic insoluble, permeable, impermeable, or biodegradable controlled release polymer, or mixtures thereof.

Most controlled release compositions or formulations are composed by two parts, a core containing active ingredient and excipient(s), and a coating. The coating material is various in its component, ratio, solubility, hydrophobic or hydrophilic. Different formulation and processing parameters can be varied in order to optimize the drug release patterns (e.g. coating level, type and amount of added plasticizer). The profile of the active ingredient releasing from the core are investigated gradually. Blends of aqueous dispersions of a water-insoluble and an enteric polymer, ethyl cellulose: hydroxypropyl methycellulose acetate succinate (EC: HPMCAS) and ethyl cellulose: methacrylic acid ethyl acrylate copolymer (EC: Eudragit L) used for pellet coating are studied (F. Siepmann et al, 2005, J. Controlled Release 105: 226-239).

One of the major problems associated with these controlled release formulations is the release of a large amount of active ingredient during the first hours following administration of the pharmaceutical formulation. This release generally results in an abrupt increase in plasma concentrations of the medicinal product, which, in many cases, results in toxicological problems which are unacceptable for humans. This “burst” release also results in a decrease in the duration of activity of the pharmaceutical composition due to the abrupt and rapid release of a large amount of said active principle subsequent to the administration of the composition.

A second problem lies in the fact that relatively inefficient encapsulation rates are generally obtained using the conventional methods of microencapsulation, in particular when the active ingredient is a water-soluble medicinal product.

A third problem which must be solved in developing these formulations is the instability of the active ingredient in the face of the rigorous conditions used in producing the microspheres, such as high temperature or prolonged contact of the active principle with an organic solvent during the solvent evaporation step.

Several trials have been carried out in order to solve these various problems. Thus, additives such as sugars, oils, wax, proteins, polymers, salts or acids have been used in the preparation of pharmaceutical compositions in the form of microspheres. These additives, which act as substances for retaining the medicinal product in the microsphere, make it possible to increase the efficiency of the method of microencapsulation and even, possibly, to protect the active principle during the process, by playing the role of stabilizing agents.

However, the inclusion of these additives in the microspheres can lead to problems of interaction between the additives and the active ingredient or the polymer-based matrix, thus inducing problems in terms of toxicology and of pharmacological activity of the medicinal product. In addition, the additives, which retain the active ingredient inside the microspheres during the production process, have an influence on the release profile of the active ingredient contained in the microspheres, possibly preventing continuous release of said active ingredient subsequent to administration of the microspheres.

Other methods of microencapsulation have also been developed in an attempt to increase the efficiency of microencapsulation of the active ingredient within the microspheres, based on the use of mixtures of organic solvents, but such methods lead to problems of stability of the active ingredient during the microsphere production process.

2-Propylpentanoic acid, more commonly known as valproic acid (VPA), its amide, valpromide (VPO), and certain salts and esters of the acid are effective in the treatment of epileptic seizures or as antipsychotic agents. U.S. Pat. No. 4,988,731 discloses an oligomer having a 1:1 molar ratio of sodium valproate and valproic acid containing 4 units, and U.S. Pat. No. 5,212,326 discloses a stable, non-hygroscopic solid form of valproic acid which comprises an oligomer having 1:1 molar ratio of sodium valproate and valproic acid and containing four to six units. Divaiproex sodium (sodium hydrogen divaiproate) is one of the most widely accepted antiepileptic agents currently available.

However, despite its efficacy in the treatment of epilepsy, valproic acid has been shown to exhibit an elimination half-life which is shorter than other commonly used anti-epileptic agents. Half-life for the drug of between six and seventeen hours in adults and between four and fourteen hours in children have been reported. This leads to substantial fluctuations in the plasma concentration of the drug, especially in chronic administration.

To overcome this disadvantage, a concerted effort has been devoted to the discovery of valproic acid formulations which will maintain more constant plasma levels of the drug following administration. The ultimate goal of these studies has been the discovery of a formulation which affords stable plasma levels in a once-a-day dosing regimen. For above reasons, a form of the active ingredient which is more slowly released to the body metabolically is essential.

The various pharmaceutical formulations of divalproex sodium are disclosed, but improvement formulations are still investigated and needed.

U.S. Pat. No. 5,009,897 discloses granules, suitable for pressing into tablets, the granules comprising a core of divalproex sodium and a coating of a mixture of a polymer and microcrystalline cellulose.

U.S. Pat. No. 5,019,398 discloses a sustained-release tablet of valproic acid: sodium valproate (1:1) in a matrix of hydroxypropyl methylcellulose, Levilite and hydrated silica, and the tablet is coated by HPMC, Eudragit E 100 and Eudragit NE 30 in the outer.

U.S. Pat. No. 5,055,306 discloses an effervescent or water-dispersible granular sustained release formulation suitable for use with a variety of therapeutic agents. The granule comprises a core comprising the active ingredient and at least one excipient, and a water insoluble, water-swellable coating comprising a copolymer of ethyl acrylate and methyl methacrylate and a water soluble hydroxylated cellulose derivative.

U.S. Pat. No. 5,169,642 discloses a sustained release dosage form comprising granules of divalproex sodium or amides or esters of valproic acid coated with a sustained release composition comprising ethyl cellulose or a methacrylic methyl ester, a plasticizer, a detackifying agent, and a slow-release polymeric viscosity agent (such as HPMC and methylcellulose).

U.S. Pat. No. 5,589,191 discloses a slow release sodium valproate tablet formulation in which the tablets are coated with ethyl cellulose containing silicic acid anhydride.

U.S. Pat. No. 6,610,326 discloses a divalproex sodium delayed-release tablet. The process for producing the tablet comprises preparing a neutralized divalproex sodium solution by combining divalproex sodium with an aqueous solvent and a base, wherein the base is used for neutralizing the valproic acid moiety of the divalproex sodium. The neutralized divalproex sodium solution is sprayed onto a pharmaceutically acceptable carrier, and processed to obtain divalproex sodium delayed-release tablets.

U.S. Pat. No. 6,419,953 discloses a hydrophilic matrix tablet suitable for the once-a-day administration of divalproex sodium, wherein the hydrophilic matrix is hydroxypropyl methylcellulose.

However, the hygroscopicity interferes and sticking of divalproex sodium is still main problems. The characteristic causes the limitation of relatively humidity during the process for producing divalproex sodium tablet. In some cases, the relatively humidity has to be maintain in 55˜60%, or even less than 30% (U.S. Pat. No. 4,913,906, U.S. Pat. No. 5,017,613, and U.S. Pat. No. 5,185,159). Such condition is unfavorable for commercializing. For the sticking of divalproex sodium, some cases disclose adding anti-sticking or sticking-preventing agent. U.S. Pat. No. 5,185,159 discloses a formulation of valproic acid and sodium valproate which is prepared without the use of either a binder or a granulating solvent. The formulation optionally contains precipitated silica as an anti-sticking or detackifying agent and the formulation is coated with polyvidone or methacrylate.

### BRIEF DESCRIPTION OF THE DRAWINGS

FIG. 1 shows the controlled release test result of 250 mg of divalproex sodium extended release tablet. (a) means the appearance of the produced tablet; (b) means the appearance of the tablet under the dissolution test for 18 hours; (c) is the cross section of the tablet under the dissolution test for 18 hours.

FIG. 2 shows the controlled release test result of 500 mg of divalproex sodium extended release tablet. (a) means the appearance of the produced tablet; (b) means the appearance of the tablet under the dissolution test for 18 hours; (c) is the cross section of the tablet under the dissolution test for 18 hours.

FIG. 3 shows the release diagram of the dissolution test of divalproex sodium extended release tablet 500 mg.

### SUMMARY OF THE INVENTION

This invention relates to a controlled release composition comprising hydrophobic cellulose, methacrylic acid copolymer and active ingredient in a form of uniform state.

This invention also relates to a method for producing controlled release tablet comprising (a) mixing hydrophobic cellulose, the methacrylic acid copolymer and active ingredient to be uniform mixture; (b) spraying hydrophobic cellulose into the mixture for forming microencapsulating granules; (c) granulation by spraying binder solution into microencapsulating granules; and (d) adding silicon dioxide, magnesium stearate, magnesium aluminum silicate into microencapsulating granules, blending and tabletting.

### DETAILED DESCRIPTION OF THE INVENTION

This invention provides a controlled release composition comprising hydrophobic cellulose, methacrylic acid copolymer and active ingredient in a form of uniform state. The hydrophobic cellulose used in the present invention is one kind of cellulose with hydrophobic ability. In a preferred embodiment, the hydrophobic cellulose is ethyl cellulose, wherein the viscosity of ethyl cellulose is from 3 to 120 mPas (cP). In a preferred embodiment, the viscosity of ethyl cellulose is from 20 to 110 mPas (cP). In a more preferred embodiment, the viscosity of ethyl cellulose is from 90 to 110 mPas (cP).

The methacrylic acid copolymer used in the present composition is methacrylic acid and methyl methacrylayte copolymer. In a preferred embodiment, the methacrylic acid and methyl methacrylayte copolymer is in a ratio of 3:1 to 1:3. In a more preferred embodiment, the ratio is 1:2 or 2:1. The commercial methacrylic acid copolymer can be Eudragit S 100.

The active ingredient of this invention can be a chemical compound, a pharmaceutical composition, or a biopharmaceutical composition. In a preferred embodiment, the active ingredient is divalproex sodium. In a more preferred embodiment, the divalproex sodium content is in the range from 25% to 55% of the weight of the composition.

For the composition of the present invention, the hydrophobic cellulose and the methacrylic acid copolymer is in a ratio of 10:1 to 1:10. In a preferred embodiment, the ratio is 3:1 to 1:3. In a more preferred embodiment, the ratio is 1.2:1. The present composition with above ratio can make a surprised result. In particular, upon contacting water or artificial intestine solution, the composition become a porous and semipermeable matrix to slowly release the active ingredient.

Because the mixtures of the active ingrendent (such as divalproex sodium) and the methacrylic acid copolymer are easy to induce the hygroscopicity, it sticks the active ingredient on the punch die during tabletting process. To solve the sticking problem, the microencapsulating technology by ethyl cellulose coating and granulation are applied to the present invention. Accordingly, the composition of the present invention further comprises ethyl cellulose for encapsulating on outer layer. The ethyl cellulose is a kind of hydrophobic cellulose, which can be used for forming a film covering the composition. In a preferred embodiment, the ethyl cellulose content for encapsulating on outer layer of the composition is in the range from 0.5% to 10% the weight of the composition. In a more preferred embodiment, the ethyl cellulose content for encapsulating on outer layer of the composition is in the range from 1% to 3% the weight of the composition.

Generally, tabletting is under less than 30% relatively humidity. It is hard to scale-up tabletting process under 40-60% relatively humidity. To overcome the hygroscopic and sticking problems under the same condition, the present composition further comprises silicon dioxide, magnesium stearate, magnesium aluminum silicate. In a preferred embodiment, the silicon dioxide content is in the range from 0.1% to 6% the weight of the composition, wherein the magnesium stearate content is in the range from 0.1% to 4% the weight of the composition, and the magnesium aluminum silicate content is in the range from 0.1% to 4% the weight of the composition.

The composition can be controlled release under fluid condition and can be used for pharmaceutical treatment for a patient.

This invention also provides a method for producing controlled release tablet comprising (a) mixing hydrophobic cellulose, the methacrylic acid copolymer and active ingredient to be uniform mixture; (b) spraying hydrophobic cellulose into the mixture for forming microencapsulating granules; (c) granulation by spraying binder solution into microencapsulating granules; and (d) adding silicon dioxide, magnesium stearate, magnesium aluminum silicate into microencapsulating granules, blending and tabletting. The present method further comprises color coating by opradry II white. In a preferred embodiment, the hydrophobic cellulose is ethyl cellulose.

In an embodiment, the methacrylic acid copolymer is methacrylic acid and methyl methacrylaytes copolymer. In a preferred embodiment, the methacrylic acid and methyl methacrylaytes is in a ratio of 3:1 to 1:3. In a preferred embodiment, the ratio is 1:1 or 1:2. In a more preferred embodiment, the ratio is 1:2.

The active ingredient of this invention can be chemical compound, pharmaceutical composition, or biopharmaceutical composition. In a preferred embodiment, the active ingredient is divalproex sodium.

In an embodiment, the hydrophobic cellulose and the methacrylic acid copolymer is in a ratio of 10:1 to 1:10. In a preferred embodiment, the ratio is 3:1 to 1:3. In a more preferred embodiment, the ratio is 1.2:1.

In a more preferred embodiment, the controlled release tablet produced by the present method comprises about 53.8% by weight divalproex sodium; about 17.7% by weight ethyl cellulose; about 14.1% by weight methacrylic acid copolymer; about 5.6% by weight microcrystalline cellulose; about 0.8% by weight polyvinylpyrrolidone; about 4% by weight silicon dioxide; about 2% by weight magnesium stearate; about 2% by weight magnesium aluminum silicate; and about 3% of the composition by weight opradry II white.

The method of the present invention can be used for producing controlled release tablet more effectively.

The features and advantages of the present invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Unless specifically stated otherwise herein, references made in the singular may also include the plural. For example, “a” and “an” may refer to either one, or one or more.

The definitions set forth herein take precedence over definitions set forth in any patent, patent application, and/or patent application publication incorporated herein by reference.

### Example

The examples below are non-limiting and are merely representative of various aspects and features of the present invention.

(Material)

The core tablet (Subtotal 1000 mg)

| | mg | % | | mg | % |
|---|---|---|---|---|---|
| Divalproex Sodium | 538.0 | 53.8 | Eudragit S-100 | 141.2 | 14.1 |
| Ethylcellulose 100 cps FP | 176.5 | 17.7 | Povidone K-30 | 8.0 | 0.8 |
| Microcrystalline cellulose | 56.3 | 5.6 | Nusilin UFL2 | 20.0 | 2.0 |
| Magnesium Stearate | 20.0 | 2.0 | Silicon dioxide | 40.0 | 4.0 |

| | | | |
|---|---|---|---|
| Ethyl alcohol | solvent | Purified water | solvent |

The color coating (Subtotal 30 mg)

| | | |
|---|---|---|
| Opradry II white (85G28725) | 30.0 mg | 3% |

| | | | |
|---|---|---|---|
| Ethyl alcohol | solvent | Purified water | solvent |

### Example 1

### Preparation of Core Tablet (10,000 Tablets)

Blending the components showed as follows:

| | |
|---|---|
| Divalprex sodium | 5380 gram |
| Ethylcellulose 100 cps FP | 1665 gram |
| Eudragit ® S-100 | 1412 gram |
| Microcrystalline cellulose | 563 gram |

Passing through a 30 mesh screen, mixing at 120 rpm for 10 minutes with supermixer and the homogenous powder was obtained.

### Example 2

### Microencapsulating Process

(a) Preparation of Microencapsulating Solution

Rx:

| | |
|---|---|
| Ethocel ® 100 cps FP | 100 gram |
| Ethyl alcohol | 1.6 liter |
| Purified water | 200 mL |

Ethyl cellulose was put into the solution containing ethyl alcohol and purified water. The solution was mixed by the stirrer until the ethyl cellulose was dissolved completely.

(b) Microencapsulating

The homogenous powders (Example 1) were put into Wurster fluid bed. The condition was set up: Inlet Temp. 70° C., Outlet Temp. 35° C., Spray rate: 40˜80 mL/minutes and pre-heat time: 5 minutes. Sparying microencapsulating solution into homogenous powder, then the microencapulation granules were available.

### Example 3

### Granulation

(a) Preparation of Binder Solution:

Rx:

| | |
|---|---|
| Povidone (K-30) | 80 gram |
| Ethyl alcohol | 320 mL |
| Purified water | 600 m |

The Povidone (K-30) was put into the solution containing ethyl alcohol and purified water. The solution was mixed by the stirrer until the Povidone (K-30) was dissolved completely.

(b) Granulation Method

The microencapulation granules (Example 2) were put into Wurster fluid bed. The condition was set up: Inlet Temp. 70° C. Outlet Temp. 35° C., Spray rate: 40˜80 mL/minutes, drying time: 5 minutes, L.O.D.<3%. Sparying the binder solution into the microencapulating granules, then the granules pass through 20 mesh screen. The divalproex sodium granules were available.

### EXAMPLE 4

### Finished Blending and Tableting

(a) Blending

Blending the components showed as follows:

| | | |
|---|---|---|
| Divalproex sodium Granules | 9200 | gram |
| Silicon dioxide | 400 | gram |
| Magnesium aluminum silicate | 200 | gram |
| Magnesium stearate | 200 | gram |
| Total: | 10,000 | gram |

(538 mg Divalproex sodium/1000 mg granules)

The above components were put into the double cone and blend for 7 minutes at 30 rpm. The divalproex sodium granules were prepared by the Step 3. Silicon dioxide, magnesium aluminum silicate and magnesium stearate were added for preventing granules adhesion in tabletting process.

(b) Tableting

The above finished blending granules were put into 20 rotating tabletting machine. The conditions were set up as follows: pre-pressure 6,000 pounds, main pressure 12,000, rotating speed 20 rpm.

The produced tablet had the specifications as follows:
Weight: 970 mg˜1030 mg (equivalent to 500 mg valproic acid activity)
Caplet shape: 18.9 mm×10 mm
Thickness: 8.3 mm˜8.8 mm
Hardness: 13 to 25 Kg

### Example 5

### Color Coating

Rx:

| | | |
|---|---|---|
| Core tablets | 10,000 | gram |
| Opradry II white (85G28725) | 300 | gram |
| Ethyl alcohol (solvent) | 970 | mL |
| Purified water (solvent) | 2265 | mL |
| total: | 10,300 | gram |

(a) Preparation of Color Coating Solution

Opradry II white was added into the solution containing ethyl alcohol and purified water. The solution was mixed by the stirrer until the Opradry II white was uniform completely.

(b) Color Coating

The core tablets from above Example 4 were put into a film coating pan. The operation condition was set up: Inlet Temp: 75˜85° C., Outlet Temp: 45˜50° C., Pan rotation: 2˜15 rpm, pre-heat time: 10 minutes, spray rate: 100 gram/minutes, drying time: 5 minutes. Spraying the color solution into the core tablets, the coated tablets were available.

The produced, tablet had weight 1030 mg (equivalent to 500 mg valproic acid activity)/per tablet.

### Example 6

### Dissolution Test

For determining the controlled release effect of the tablet, the dissolution test was performed.

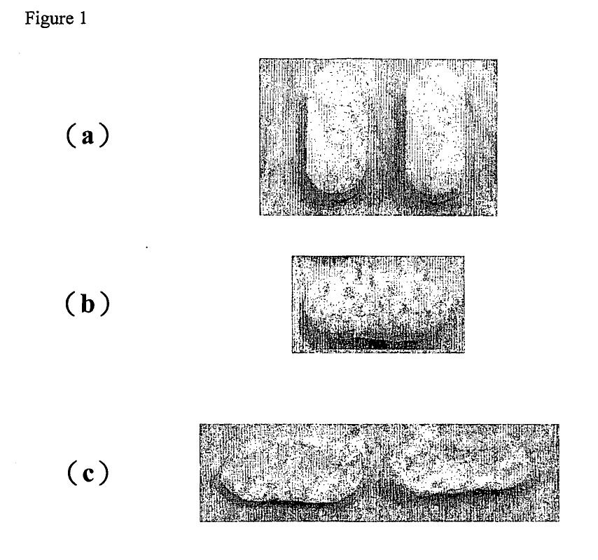

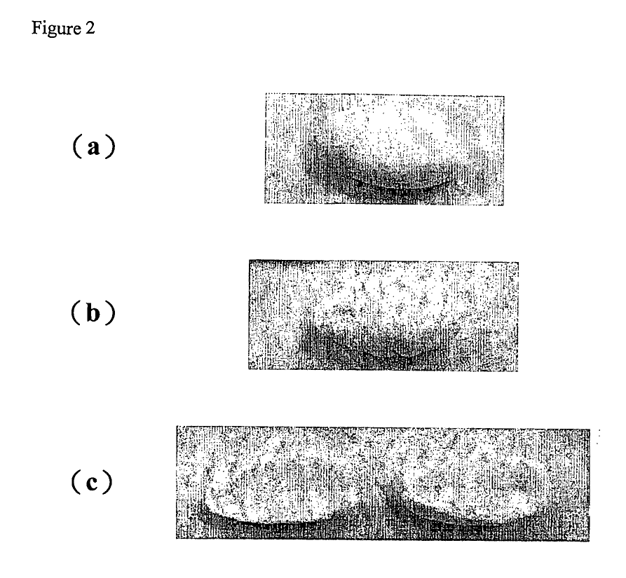

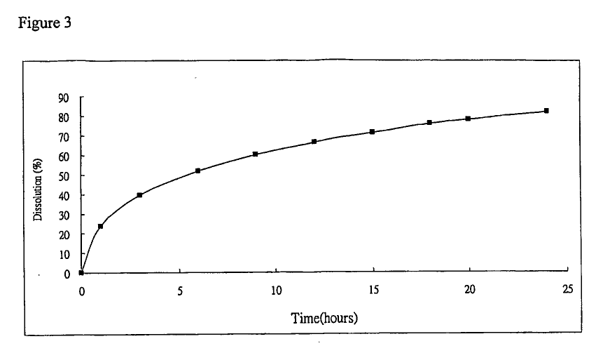

The divalproex sodium extended release tablet 250 mg and 500 mg were tested. The dissolution condition was in pH 6.8 phosphate buffer medium, 100 rpm stir speed, in apparatus Paddle, for 18 hours. The tablet photos were shown in FIG. 1 and FIG. 2, including the tablet before (FIG. 1a and FIG. 2a) and after (FIG. 1b and FIG. 2b) the test. The cross sectional drawings were also showed in FIG. 1c and FIG. 2c. The FIG. 3 was the release diagram of dissolution test of the divalproex sodium extended release tablet 500 mg. According to the result of dissolution test, it was clear showed that the tablet had well controlled release effect.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

What is claimed is:

1. A controlled-release composition comprising hydrophobic cellulose, methacrylic acid copolymer and active ingredient in a form of uniform state.

2. The composition according to claim 1, wherein the hydrophobic cellulose is ethyl cellulose.

3. The composition according to claim 2, wherein the ethyl cellulose has viscosity from 3 to 120 mPas (cP).

4. The composition according to claim 3, wherein the ethyl cellulose has viscosity is 90 to 110 mPas (cP).

5. The composition according to claim 1, wherein the methacrylic acid copolymer is methacrylic acid and methyl methacrylayte copolymer.

6. The composition according to claim 5, wherein the methacrylic acid and methyl methacrylayte is in a ratio of 3:1 to 1:3.

7. The composition according to claim 5, wherein the ratio is 1:1 or 1:2.

8. The composition according to claim 1, wherein the active ingredient is divalproex sodium.

9. The composition according to claim 8, wherein the divalproex sodium content is in the range from 25% to 55% of the weight of the composition.

10. The composition according to claim 1, wherein the hydrophobic cellulose and methacrylic acid copolymer is in a ratio of 10:1 to 1:10.

11. The composition according to claim 1, wherein the hydrophobic cellulose and methacrylic acid copolymer is in a ratio of 3:1 to 1:3.

12. The composition according to claim 1, wherein the ratio of the hydrophobic cellulose and methacrylic acid copolymer is 1.2:1.

13. The composition according to claim 1, which further comprises ethyl cellulose for encapsulating on outer layer of the composition.

14. The composition according to claim 13, wherein the ethyl cellulose content for encapsulating on outer layer of the composition is in the range from 0.5% to 10% the weight of the composition.

15. The composition according to claim 13, wherein the ethyl cellulose content for encapsulating on outer layer of the composition is in the range from 1% to 3% the weight of the composition.

16. The composition according to claim 1, which further comprises silicon dioxide, magnesium stearate and magnesium aluminum silicate.

17. The composition according to claim 16, wherein the silicon dioxide content is in the range from 0.1% to 6% the weight of the composition, the magnesium stearate content is in the range from 0.1% to 4% the weight of the composition, and the magnesium aluminum silicate content is in the range from 0.1% to 4% the weight of the composition.

18. A method for producing a controlled release tablet comprising:
(a) blending hydrophobic cellulose, methacrylic acid copolymer and active ingredient to be uniform mixture;
(b) spraying hydrophobic cellulose into the mixture for forming microencapsulating granules;
(c) granulation by spraying binder solution into microencapsulating granules;
(d) adding silicon dioxide, magnesium stearate, magnesium aluminum silicate into microencapsulating granules, blending and tabletting;

19. The method of claim 18, which further comprises color coating by opradry II white.

20. The method of claim 18, wherein the hydrophobic cellulose is ethyl cellulose.

21. The method of claim 18, wherein the methacrylic acid copolymer is methacrylic acid and methyl methacrylayte copolymer.

22. The method of claim 21, wherein the methacrylic acid and methyl methacrylayte is in a ratio of 3:1 to 1:3.

23. The method of claim 21, wherein the methacrylic acid and methyl methacrylayte ratio is 1:2.

24. The method of claim 18, wherein the active ingredient is divalproex sodium.

25. The method of claim 18, wherein the hydrophobic cellulose and methacrylic acid copolymer is in a ratio of 10:1 to 1:10.

26. The method of claim 18, wherein the tablet comprises about 53.8% by weight divalproex sodium; about 17.7% by weight ethyl cellulose; about 14.1% by weight methacrylic acid copolymer; about 5.6% by weight microcrystalline cellulose; about 0.8% by weight polyvinylpyrrolidone; about 4% by weight silicon dioxide; about 2% by weight magnesium stearate; about 2% by weight magnesium aluminum silicate; and about 3% of the composition by weight opradry II white.
